(19) United States
(12) Patent Application Publication
(10) Pub. No.: US 2026/0017797 A1
(43) Pub. Date: Jan. 15, 2026

(54) COMPUTER-IMPLEMENTED METHOD FOR EVALUATING IMAGE DATA DEPICTING A VASCULAR STRUCTURE, MEDICAL IMAGING DEVICE, COMPUTER DEVICE AND STORAGE UNIT

(71) Applicant: Siemens Healthineers AG, Forchheim (DE)

(72) Inventor: Annette Birkhold, Stuttgart (DE)

(21) Appl. No.: 19/264,247

(22) Filed: Jul. 9, 2025

(30) Foreign Application Priority Data
Jul. 10, 2024 (DE) 10 2024 206 513.7

(51) Int. Cl. G06T 7/00; A61B 6/00; A61B 6/03; A61B 6/50; G06T 7/11; G06T 7/73

(52) U.S. Cl. CPC G06T 7/0016; A61B 6/032; A61B 6/4441; A61B 6/481; A61B 6/504; G06T 7/11; G06T 7/73; G06T 2200/04; G06T 2207/10081; G06T 2207/10088; G06T 2207/20072; G06T 2207/30104

(57) ABSTRACT

A computer-implemented method for evaluating image data depicting a vascular structure for the purpose of automatically determining at least one arterial and/or venous access position of the vascular structure. An embolization may be carried out at at least one peripheral position of a pathological change in the region of the vascular structure by introducing a catheter via an access path which runs through the vascular structure and leads from the access position or one of the access positions to the respective peripheral position. The method includes obtaining the image data comprising a plurality of image points, wherein those image points through whose respectively associated position in the vascular structure a contrast agent flowed during the recording of the image data, or of raw images from which the image data is derived, are each assigned at least one captured item of time information relating to a time point at which the contrast agent reached the respective position, the contrast agent being administered during or before this recording and determining the at least one arterial and/or venous access position on the basis of the at least one item of time information.

### CROSS REFERENCE TO RELATED APPLICATIONS

This application claims the benefit of DE 10 2024 206 513.7 filed on Jul. 10, 2024, which is hereby incorporated by reference in its entirety.

### FIELD

Embodiments relate to a computer-implemented method for evaluating image data depicting a vascular structure for automatically determining at least one arterial and/or venous access position of the vascular structure.

### BACKGROUND

Pathological changes, for example arteriovenous malformations, respective deformities or tumors, are frequently treated by so-called embolization. In this context, blood vessels which form an access to or an issue from the respective change are artificially closed by a catheter, for example by inserting plastic parts or fibrin sponges. For the purpose of carrying out the embolization, the catheter must be brought to the position at which the embolization is to take place via a corresponding access.

When preparing and carrying out the embolization, of critical significance is the knowledge of the geometric conditions relating to the vascular structure in the region of which the pathological change is present, or which is affected by the pathological change. The knowledge of these conditions relating to the access path that runs through the vessels of the vascular structure is therefore required for the catheter. Corresponding vascular structures, for example of the brain, differ from patient to patient and typically have complex geometries, for example branching vessels. In the region of the brain for example, the local vascular structure may also be referred to as a vascular tree. The vessels therefore branch out continuously, starting from an arterial carotid artery. They eventually reconnect again continuously to form one or more veins in the venous region.

A known approach when preparing for an embolization is so-called segmentation of the image data. In the context of medical imaging, this is understood to mean a process whereby specific structures or regions within an image are identified, for example the pathological change. The segmentation is helpful when carrying out the embolization, as the course of the blood vessels to the respective structure may be traced more effectively and more easily thereby. An example relating to the actual implementation of such a segmentation is described in the currently unpublished German patent application having the official application number 10 2023 211 997.8.

### BRIEF SUMMARY AND DESCRIPTION

The scope of the embodiments is defined solely by the appended claims and is not affected to any degree by the statements within this summary. The present embodiments may obviate one or more of the drawbacks or limitations in the related art. Independent of the grammatical term usage, individuals with male, female or other gender identities are included within the term.

Embodiments provide an improved concept in connection with determining an arterial and/or venous access position of the vascular structure for the purposes of an embolization that must be carried out.

Embodiments provide a method including the following steps: obtaining the image data including a plurality of image points, those image points through whose respectively associated position in the vascular structure a contrast agent flowed during the recording of the image data, or of raw images from which the image data is derived, each being assigned at least one captured item of time information relating to a time point at which the contrast agent reached the respective position, determining the at least one arterial and/or venous access position on the basis of the at least one item of time information.

The contrast agent is administered during or before the recording of image data or raw images. The administration of the contrast agent is however not an element or method step. In order to carry out the method, it is merely necessary for a contrast agent to flow through the vascular structure in question.

By the method, with reference to the image data and with additional use of the item of time information, the knowledge of the access position that is required for the purpose of carrying out the embolization may be determined as reliably and accurately as possible. This is based on the assumption that the contrast agent initially flows through the arterial vessels of the vascular structure and then through the blood vessels branching therefrom, ultimately reaching the veins. By using the at least one item of time information, it is thereby possible to distinguish between the respective arteries and veins. This information is useful for example because arteries and veins have different properties, for example in respect of elasticity, and this information is possibly significant when carrying out the embolization.

The image data or the raw images may be present in each case as a two-dimensional image including a plurality of pixels or as a three-dimensional image including a plurality of voxels. The image data or the raw images as supplemented by the item of time information therefore form a three-dimensional or four-dimensional data set including two or three spatial dimensions and one time-related dimension. The image data or the raw images are available as at least one two-dimensional or three-dimensional array of pixels or voxels. Such arrays of pixels or voxels may represent color, intensity, absorption or other parameters as a function of the two-dimensional or three-dimensional position. They may be obtained by processing measured signals from a medical imaging device in an appropriate manner, for example.

The image data or the raw images may be present as at least one angiographic image data set or include at least one such image data set. Angiographic image data is image data that represents a blood vascular system of a patient or a body part of a patient. An angiographic image data set contains image data of the blood vascular system or of the vascular structure. The image data or the raw images may be stored in a standard image format such as the Digital Imaging and Communications in Medicine (DICOM) format, and saved in a storage system or computer storage system such as a Picture Archiving and Communication System (PACS), Radiology Information System (RIS), etc.

The administration or injection of the contrast agent takes place when capturing the image data or the raw images. The vascular structure may be identified more clearly as a result of the contrast agent. The contrast agent may be injected into an arterial vessel that leads to the vascular structure. Following the injection, and as a result of the blood flow, the contrast agent arrives at the vascular structure and flows through it. The item of time information or one of the items of time information may relate to or specify the time point at which the contrast agent first reaches the respective position.

In relation to the capture of the image data or the raw images, these may be obtained by performing an image subtraction of corresponding raw recordings. As part of this activity, sections that coincide are mapped for the purpose of capturing the raw recordings, a so-called mask being captured first, i.e. a raw recording in which the contrast agent has not yet reached the vascular structure. Further raw recordings are then captured in which the contrast agent flows though the vascular structure. For the purpose of generating the image data or the raw images, the mask is subtracted from the other raw recordings in order to eliminate structures that are not relevant for the present examination, for example bones or similar, from the image data. As a result, only the vascular structure filled with contrast agent then remains as image information. This procedure is also referred to as digital subtraction angiography.

In relation to the capture of the image data or the raw images and the at least one item of time information, a plurality of raw images may be captured in chronological sequence over a time period during which the contrast agent flows into the vascular structure. Therefore, for the various raw images, various situations are recorded relating to those parts of the vascular structure through which the contrast agent has already flowed. The item of time information is obtained therefrom.

For the purpose of capturing the image data or the raw images, a medical imaging method may be carried out using computed tomography. In relation to the capture of the image data or the raw images, it is possible to use any imaging modality suitable for this, for example for the purpose of generating angiographic image data, for example an x-ray system, for example the cited computed tomography system. Computed tomography is a widely used imaging method that uses x-rays that are generated and captured by an instrument that physically rotates around the patient. This may take the form of so-called cone-beam computed tomography (CBCT), that is based on images from a conical x-ray beam, such as those generated by a C-arm x-ray system. Or it may take the form of fan-beam computed tomography, that is based on images from a fan-shaped x-ray beam, such as those generated by a computed tomography system, for instance. The resulting absorption data is processed by computer-aided analysis software that reconstructs detailed images, for example three-dimensional images, of the internal structure of the body parts of the patient. The generated image data sets are referred to as CT scans, that may represent a plurality of series of sequential images in order to show the internal anatomical structures in cross sections perpendicular to the axis of the human body.

In the context of computed tomography, two-dimensional raw images are captured in chronological sequence while the contrast agent flows into the vascular structure. As mentioned above, an analysis of the positions to which the contrast agent has progressed in the vascular structure at the respective time point allows the at least one item of time information to be established. From the two-dimensional raw images, a processing device then calculates a three-dimensional raw image that represents the image data or is used to establish the image data.

If the medical imaging method for capturing the image data is carried out using computed tomography, an angular range of more than 180° may be scanned by a C-arm for the purpose of carrying out the computed tomography, spatially three-dimensional image data or at least one spatially three-dimensional raw image being derived from a set of two-dimensional raw images obtained in the course of this scan. An angular range of 180° is typically scanned for the purpose of capturing a three-dimensional data set, the range here being correspondingly extended because for example the item of time information is additionally captured.

For the purpose of capturing the image data or the raw images, a medical imaging method may be carried out using magnetic resonance tomography. Magnetic resonance tomography (MRT) is an advanced medical imaging technique that uses the effect of the magnetic field on the movements of protons. In the case of MRT devices, the detectors are antennas and the signals are analyzed by a computer that produces detailed images of the internal structures in each section of the human body

The raw images may be segmented for the purpose of generating the image data. As mentioned in the introduction, the segmentation allows the identification of the pathological region to take place. The raw images or the segmented image data may be converted into a graph that includes a collection of nodes and arcs and that models the vascular structure. A graph is understood to be an abstract structure that represents a collection of objects together with the connections that exist between these objects. The mathematical abstractions of the objects are referred to as nodes in this case. Insofar as the image data is available as the at least one graph, the nodes may be understood as the image points. The paired connections between nodes are referred to as arcs. In this sense, an arc is a direct connection between two nodes. A graph may be shown clearly by representing the nodes as points and the arcs as lines. The segmentation takes place first, followed by the generation of the at least one graph. In the context of performing the segmentation, a region that may be assigned to the pathological change is identified within the image data or the raw images, it being possible with reference to the at least one graph derived therefrom to identify at least one node that is arranged in the peripheral region of this region as the at least one peripheral position. For this purpose, it is possible to proceed in accordance with the German patent application 10 2023 211 997.8 cited above, the disclosure therein being included by reference in the present technical teaching correspondingly.

Embodiments provide, for those image points whose corresponding position in the vascular structure is captured by the contrast agent, checking whether an artery condition is met, those image points for which the artery condition is met being identified as an arterial access position or as belonging to an arterial access position, the artery condition being met or only able to be met if the item of time information or at least one of the items of time information implies that an arrival time of the contrast agent at the corresponding position is less than an arterial time limit value. The item of time information may be present as a numerical value; it being checked in this case whether the respective numerical value is less than the arterial time limit value. The arterial time limit value may be permanently specified or imposed individually for the respective procedure. It is therefore possible with reference to the image data to determine the time point at which the contrast agent in the vascular structure first arrives at a branch location, this time point representing the arterial time limit value.

It is similarly possible, for those image points whose corresponding position in the vascular structure is captured by the contrast agent, to check whether a vein condition is met, those image points for which the vein condition is met being identified as a venous access position or as belonging to a venous access position, the venous condition being met or only able to be met if the item of time information or at least one of the items of time information implies that an arrival time of the contrast agent at the corresponding position is greater than a venous time limit value. The item of time information may be present as a numerical value; it being checked in this case whether the respective numerical value is greater than the venous time limit value. The venous time limit value may be permanently specified or imposed individually for the respective procedure.

The situation relating to the venous time limit value is essentially more complex than that relating to the arterial time limit value. This is because the time before the contrast agent arrives at the venous access position may vary for different flow paths through the vascular structure. In consideration of this for example, it may be advantageous to capture a plurality of items of time information for each of the image points, for example relating to the first arrival time of the contrast agent at the respective position, and indeed specifically for different flow paths through the vascular structure.

For example given this circumstance that complicates the identification of the venous access position, and in order that reliable results may be achieved as far as possible in relation to this, embodiments provide that the artery condition and/or the vein condition at the respective image point is only met or may only be met if, in addition to the temporal condition relating to the respective arrival time of the contrast agent, a locality condition is met that is only met or may only be met if the respective image point may be assigned to a collection of a plurality of image points, these being arranged directly adjacent to each other, for whom the temporal condition is likewise met, and if the collection of the image points implies that this represents a blood vessel having a geometric extent that is greater than a specified limit extent. Therefore, a resolution of the image data is typically high enough for the vessel forming the respective access position in the image data to be depicted by a plurality of adjacently situated image points, i.e. for example pixels or voxels or nodes. The geometric shape of the collection including the corresponding image points reflects the geometric shape of the respective blood vessel. For example, the dimensions of this shape therefore also correlate to the dimensions of the respective blood vessel. For the purpose of identifying the access position, the knowledge is used of the fact that corresponding arteries or veins typically have a certain minimum size. In the case of smaller dimensions, it must be assumed that the respective vessels are already branches of the vascular structure and therefore are not corresponding access positions. Specifically, it is possible to check whether the depicted blood vessel has a predetermined minimum diameter. Regarding this, an evaluation may also take place in respect of the actual geometric shape of the depicted vessel.

According to an embodiment, a plurality of data sets including image data are obtained and that for the purpose of obtaining the image data of one of the data sets, the injection of the contrast agent takes place via a blood vessel assigned to the respective data set. In this context, provision may advantageously be made for the injection of the contrast agent to take place in different blood vessels for different data sets. In principle, each of the arteries leading to the vascular structure represents a candidate for the access position. The injection of a contrast agent into various blood vessels or arteries therefore results in the various image data of various data sets in each case, it being possible to identify the corresponding candidate in each case with reference to the various data sets. Furthermore, a more substantial database is also thereby established in relation to any access paths formed by veins, for example in order to achieve useful results in spite of the complex situation described above in relation to the venous time limit value. The image data of the data sets may therefore be evaluated as part of a corresponding data merger.

In relation to the at least one item of time information, the time point of the injection of the contrast agent may be used as a reference time point for the at least one item of time information. The corresponding item of time information may therefore be present as a value that specifies the time elapsed since the injection of the contrast agent, for example in seconds. With reference to the embodiment described above, for the image data of each of the data sets, the time point of the respective injection of the contrast agent may be used as a reference time point for the at least one item of time information. On the basis of the image data of the data sets, it is then possible to identify an image point that depicts a common location in the vascular structure, the item of time information assigned to this image point being used as a reference time point for the image data of the respective data set. In relation to the values for the item of time information, this reference time point may therefore be defined as a zero point in the context of a corresponding transformation. The transformation may therefore take place in such a way that a constant time value is subtracted from each of the time values, the constant time value being the item of time information that is assigned to the image point that depicts the shared location in the vascular structure. This makes it possible for the data sets to be compared and consolidated or combined.

In addition to determining the at least one access position, at least one route that leads through the vascular structure and connects the respective access position to the respective peripheral position may be established as the at least one access path. At least one route via which the catheter, starting from the access position, may be guided to the peripheral position, i.e. that position at which the embolization must take place, is therefore established as the access path. It is conceivable to establish a plurality of routes that represent possible candidates for corresponding access paths. In this context, the final decision as to which of the established routes is ultimately used when carrying out the embolization is made by the acting doctor.

Embodiments may establish as the at least one access path a route that leads through the vascular structure and along whose course a diameter of the blood vessel concerned is greater than a specified minimum diameter and/or a curvature of the route locally is smaller than a maximum curvature. In other words, with regard to establishing the at least one access path, it is possible to establish a plurality of routes that are theoretical candidates for this. In order to determine whether the respective route is actually a possible access path, it may be checked whether at least one corresponding suitability criterion is met. It is thus possible to perform a preselection, for example an automatic preselection, in relation to this. The suitability criterion relates to the question of whether, based on geometric conditions, the respective route is fundamentally suitable to be used as the access path for the catheter. The suitability criterion may relate to the geometry of the route or the vessels leading along the route. For example, the suitability criterion might conceivably only be met if the route that could possibly represent an access path is formed by blood vessels of the vascular structure whose diameter is greater than the specified minimum diameter. Furthermore, the suitability criterion might only be met if the vessels forming the respective route do not at any location include a curvature that is greater than the specified maximum curvature.

On the basis of the results obtained by the method, for example on the basis of the segmented graph including the at least one access position and the at least one peripheral position and the at least one access path, a sectional view may be generated in which the results are inserted via superimposition or presented in a highlighted manner. In terms of its presentation region, the sectional view may correspond to a live image that, during the performance of the embolization, is recorded by a medical imaging device and is displayed to the acting doctor in real time, this sectional view may be inserted into the live image. This makes it easier for the doctor to guide the catheter, starting from the respective access position, via the access path to the peripheral position.

At least some of the steps of the method may be carried out by artificial intelligence in the form of software. In comparison with determination by a human of the at least one access position, the use of artificial intelligence produces an efficiency gain in respect of the required resources, for example time. A further advantageous result is that artificial intelligence makes it possible to consider manifold details and interrelationships that the human user is not capable of assessing. The result of the evaluation of the image data consequently has the greatest chance of success in respect of the purpose of performing the embolization.

The artificial intelligence may be provided as a trained model that may be used in the course of performing the method. For the purpose of training the model, provision may be made for at least one set of training image data together with a training result that is assigned to the at least one set of training image data respectively, to be predetermined. The model may be trained on the basis of the at least one set of training image data and the training result, whereby the trained model is obtained. So-called machine learning is therefore carried out. The model realizes cognitive functions that correspond to or at least resemble the standard model of human thought. As a result of carrying out the training, the model is essentially capable of revealing and utilizing previously unidentified interrelationships and patterns. The determination of sizes and/or circumstances and/or interrelationships by the model may therefore be further developed and improved by performing the training. The training involves the sequential performance of discrete training steps or cycles, for example using a plurality of sets of training image data successively so that the model is continuously improved. Specifically, supervised training may be carried out, though unsupervised training may also be used.

Real image data that already exists may be used as the training image data. If supervised training is performed, results that are found useful and identified by the user from the training, and that relate to the at least one access position and/or the at least one peripheral position and/or the at least one access path and/or the region that is determined by the segmentation and represents the pathological change, may be specified as training results that represent ideal solutions. The results that are generated in the context of the training by the model may be compared with these, an objective being to minimize the variations between the training results and the generated results. When using a model that is based on a neutral network, the training image data is supplied via an input layer of the model, the results of the evaluation being provided via an output layer of the model. A large number of further layers may be present between these, each including a certain number of nodes between that are formed connections for realizing a neural network in the context of the training. Details relating to this are sufficiently well known to a person skilled in the art and are not set forth in greater detail as they do not affect the main aspect of the present invention.

Embodiments further relates to a medical imaging device including at least one imaging unit that is configured to provide the image data and at least one computer device that is configured to carry out the evaluation of the image data in accordance with the method as described above. By the imaging unit, it is possible for example to carry out the computed tomography or magnetic resonance tomography mentioned above. The computer device may be a system including a storage entity, a working memory, one or more microprocessors and one or more input/output data interfaces. All of the advantages, features and aspects explained in connection with the method may equally be applied to the imaging device and vice versa.

Embodiments further relate to a computer device for a medical imaging device including a storage unit on which is stored an executable computer program that is suitable to be read by a processing device of the computer device and then to cause the processing device to execute the steps of the method according to the description above. The storage unit may be understood to be a unit that is capable of storing data and information. The storage unit may be realized as any type of storage medium, and may be provided for example as a hard disc, a solid state drive, an optical drive, a magnetic drive, a flash memory or other type of non-volatile or volatile memory. All of the advantages, features and aspects explained in connection with the method and the imaging device may equally be applied to the computer device and vice versa.

Embodiments further relate to a storage unit for a computer device on which storage unit is stored an executable computer program that is suitable to be read by a processing device of the computer device and then to cause the processing device to execute the steps of the method according to the description above. All of the advantages, features and aspects explained in connection with the method, the imaging device and the computer device may equally be applied to the storage unit and vice versa.

### BRIEF DESCRIPTION OF THE FIGURES

FIG. 1 depicts an medical imaging device in accordance with an embodiment, including an computer device in accordance with an embodiment, having an storage unit in accordance with an embodiment.

FIG. 2 depicts a flow diagram of an method according to an embodiment.

FIG. 3 depicts a diagram of a raw image that is obtained while carrying out the method according to an embodiment.

FIG. 4 depicts a diagram of image data in the form of a graph according to an embodiment.

FIG. 5 depicts a perspective of an graph with established access positions according to an embodiment.

FIG. 6 depicts a perspective of results according to an embodiment.

### DETAILED DESCRIPTION

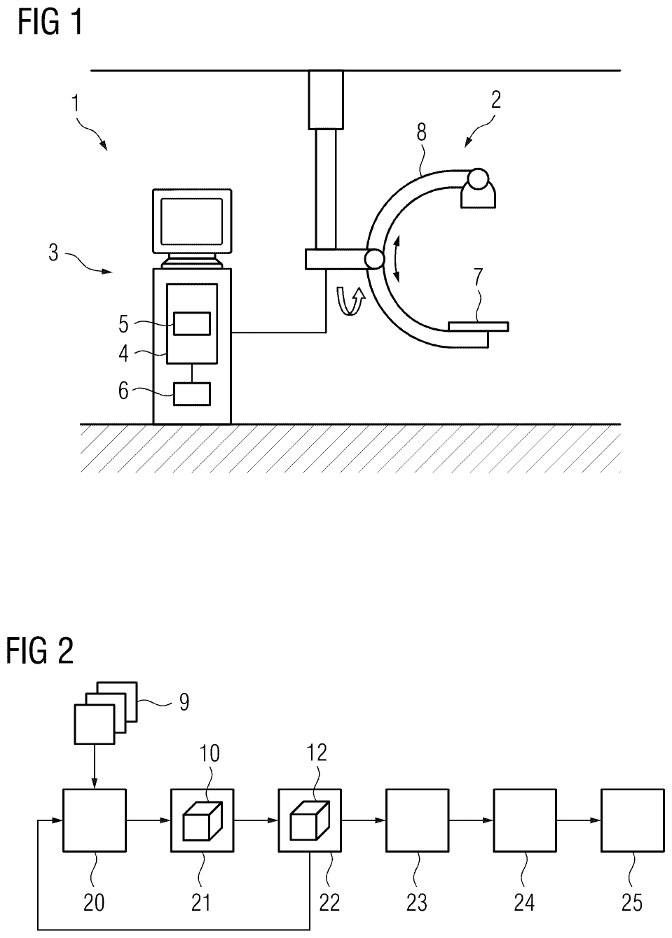

FIG. 1 depicts a schematic elementary diagram of an medical imaging device 1 in accordance with an embodiment, with reference to which an method in accordance with an embodiment is explained below, the method including the steps 20 to 25. The imaging device 1 includes an imaging unit 2, which by way of example here is a computed tomography system in the form of a C-arm x-ray system, by which it is possible to capture computed tomography recordings. By way of example, angiographic image data may be generated as raw images by the imaging unit 2, it being also possible to use, for example, a magnetic resonance tomography system. The imaging device 1 further includes an computer device 3 in accordance with an embodiment, including an storage unit 4 in accordance with an embodiment, on which is stored an executable computer program 5. A processing device 6, for example a processor, of the computer device 3 is configured to read the computer program 5, whereby the processing device 6 is induced to execute some of the method steps explained below. The processing device 6 is therefore configured to process the data or recordings that are captured by the imaging device 2.

By way of example, it is assumed in the present embodiments that a patient suffers from an arteriovenous malformation in the region of the brain. This is a pathological change 11 relating to a deformity of the blood vessels here, where arterial or blood-supplying blood vessels are connected to venous or blood-removing blood vessels in a network of an affected vascular structure 16. An evaluation of recordings here is often prone to difficulties, for example because a clear demarcation between the deformed vascular system and the healthy part of the vascular structure 16 is often hard to ascertain due to the arteriovenous malformation appearing as a complex tangle of vessels on corresponding images. Precisely this aspect is however particularly important with regard to the treatment of the pathological change 11.

An embolization is therefore envisaged as a treatment for this, whereby at peripheral positions 15 of the pathological change 11 the blood vessels forming the inflows or outflows of the pathological change 11 are closed by a catheter. For this purpose, for example the knowledge of access positions 17, 18 of the vascular structure 16 is crucially important, since the catheter is inserted into the vascular structure 16 via these access positions 17, 18 and then pushed forwards to the respective peripheral position 15. Concerning the access positions 17, 18, it is often necessary to know whether the respective access position 17, 18 is an artery or a vein. The present method is sometimes used for the purpose of determining and identifying arterial access positions 17 and venous access positions 18 of the vascular structure 16, and allowing a reliable distinction to be made between these two cases, on the basis of the evaluation of the recordings.

The embodiment of the method is explained in the following with reference to the flow diagram illustrated in FIG. 2. The first step 20 of the method therefore assumes a situation immediately after the injection of a contrast agent into an artery leading to the vascular structure 16 of a patient lying on a patient couch 7 of the imaging unit. The first step 20 therefore involves the acquisition of two-dimensional raw images 9 by the imaging device 2. For the purpose of capturing the raw images 9 by a C-arm 8, the imaging device 2 therefore scans an angular range greater than 180°, for example 270°. The raw images 9 obtained here form a two-dimensional array of pixels in each case.

With regard to the raw images 9, a mask is recorded before the contrast agent reaches the vascular structure 16. This mask is subtracted from the raw images 9 that are recorded successively while the contrast agent flows into the vascular structure 16. Structures such as bones or the like are eliminated from the resulting raw images 9. As a result, the remaining raw images 9 only show the blood vessels that are filled with contrast agent and therefore the vascular structure 16 or at least the corresponding part of the vascular structure 16.

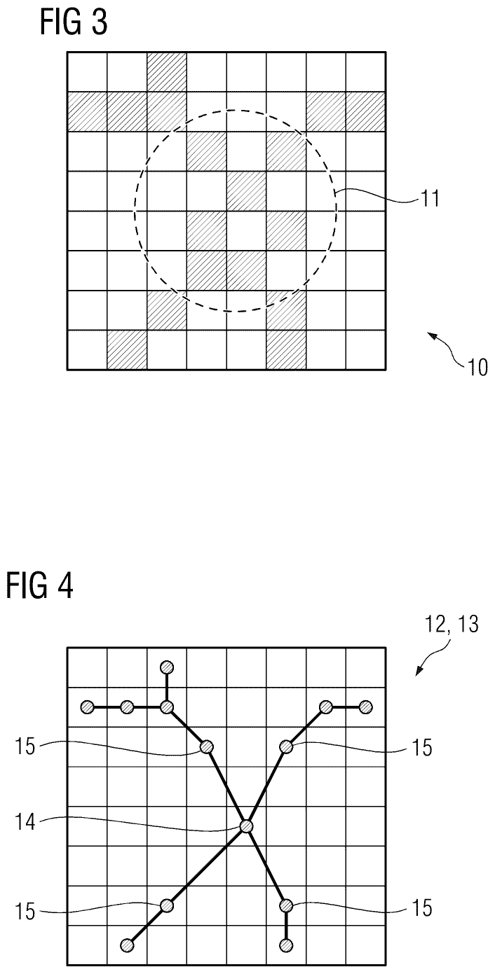

During the course of the next step 21, the two-dimensional raw images 9 are converted by the processing device 6 into a three-dimensional raw image 10 forming a three-dimensional array of voxels. During the course of the first two steps 20 and 21, a so-called digital subtraction angiography therefore takes place. FIG. 3 depicts a diagram of the raw image 10 that, for the sake of simplicity, is represented in a rudimentary two-dimensional image grid. The region of the pathological change 11 is indicated by an oval.

In the next step 22 of the method, in addition to automated segmentation, i.e. an identification of the pathological change 11 in the raw image 10, a conversion of the raw image 10 into image data 12 take place. The image data 12 is then available as a graph 13. This procedure is effected by the processing device 6 and, for example, in accordance with the description in the patent application 10 2023 211 997.8 cited above which is incorporated by reference in its entirety.

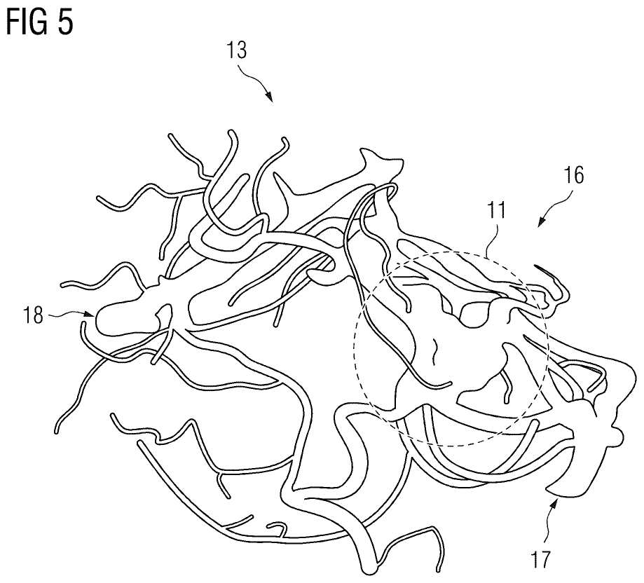

The result of the step 22 is the graph 13 shown in FIG. 4, that is derived from the three-dimensional raw image 10 and has the same grid as the raw image 10. The graph 13 includes nodes, represented as points, and arcs that connect these nodes together. As a result of performing the segmentation known from the prior art, one of the nodes is identified as a central node 14 that is understood to be a center of the pathological change 11 and may be referred to as a nidus, and the other nodes are identified as the peripheral positions 15.

A further aspect concerning the raw data 9 and the image data 12 relates to the fact that, in addition to the spatial coordinates, an item of time information is also assigned to the image points, i.e. the pixels, voxels and nodes. Specifically, the item of time information is a numerical value that specifies the time that elapsed since the administration of the contrast agent and until the contrast agent reached the respective position in the vascular structure 16.

Before explaining the further procedure relating to this, reference is made to FIG. 4, that depicts the three-dimensional spatial graph 13 in a less simplified illustration than FIG. 4. In the vascular structure 16 that may be recognized here, marked in addition to the pathological region 11 are an arterial access position 17 and a venous access position 18, these being identified by the processing device 6 in accordance with the explanation later in the text.

After the method steps 20 to 22 as explained above have been carried out for the first time, together with the associated capture of a first data set including the raw images 9, 10 and the generation of the image data 12 or the graph 13, these steps are carried out again. As part of this activity, a second data set including the raw images 9, 10 is established, and corresponding image data 12 or the graph 13 is established therefrom. The second data set differs from the first data set in that the contrast agent was administered via a different artery for the purpose of capturing the raw images 9, 10. For example by virtue of the vascular structure 16, for example in the case of the brain, having a plurality of arterial access positions and/or venous access positions, the administration of the contrast agent via various vessels allows the identification of same. Since two arteries having downstream issue are present in the case of the brain, the steps 20 to 22 are executed twice accordingly and the contrast agent is injected into one of these two arteries respectively in the context of the two executions. It applies that if further arteries leading to the vascular structure 16 are present, then the steps 20 to 22 may be executed multiple times accordingly.

In the next step 23, the image data 12 from the captured data sets is merged by the processing device 6. A separate graph 13 therefore exists as corresponding image data 12 from each execution, the graph 13 including corresponding nodes and arcs as three-dimensional information and the at least one item of time information that is assigned to each of the nodes. As discussed previously, a numerical value that specifies the arrival time of the contrast agent at the corresponding location is provided in each case as the item of time information. For each of the graphs 13, a separate reference time point is therefore used in relation to the item of time information in each case, specifically the time point at which the contrast agent is administered.

In order to allow the image data 12 from various executions to be compared and combined, a transformation in relation to the item of time information takes place in the image data 12 of each of the data sets. For this purpose, a common location in the vascular structure 16 is identified in the respective graph 16 for each of the data sets, the location referring to the same position in the vascular structure 16 in the graphs 16 of all data sets. The numerical value that exists in each case, and represents the item of time information for the image point or node, is used as a new reference time point for this purpose. Therefore, in the image data 12 of each of the data sets, the respective new reference time point or the corresponding numerical value is subtracted from all other numerical values representing the item of time information in the respective image data 12.

Therefore the result of the step 23 is, for example, a set of the image data 12 or graphs 13 that were obtained during the course of the repeated execution of the steps 20 to 22, and that were transformed in relation to the item of time information as explained above. It is however also conceivable for the image data 12 to be updated to the effect that a new graph 13 is determined, in which a plurality of items of time information, specifically the transformed items of time information, are assigned to each image point. In relation to the spatial information or coordinates for the image points, the result of one of the executions may be used in the new graph 13. Also conceivable in relation to this is a summary of the spatial information or coordinates, for example an average.

In the next step 24, the actual identification of the access positions 17, 18 is performed by the processing device 6 and with reference to the image data 12 or the graph 13. This involves checking, for each of the nodes and with reference to the respective item of time information, whether an artery condition and a vein condition are met. If the artery condition is met, it is assumed that the location in the vascular system 16 corresponding to the respective image point is an arterial access position 17. If the vein condition is met, it is assumed that the location in the vascular system 16 corresponding to the respective image point is a venous access position 18.

The artery condition may only be met if a temporal condition is met, this being the case if the item of time information assigned to the respective image point specifies that the arrival time of the contrast agent at the corresponding position is less than an arterial time limit value. The arterial time limit value, that may be permanently specified in principle, is imposed individually in the present case. With reference to the image data 12, the time point is therefore determined at which the contrast agent in the vascular structure 16 first arrived at a branch location, this time point representing the arterial time limit value. With reference to the FIG. 5, this check depicts that the artery condition is met for all voxels that are present in the region of the arterial access position 17.

Furthermore, in relation to checking the artery condition of all image points for which the temporal condition is met as explained above, it is additionally checked whether a locality condition is met. The locality condition is met if the respective image point may be assigned to a collection of a plurality of image points that are arranged directly adjacent to each other and for which the temporal condition is likewise met. Moreover, in order to meet the locality condition, the collection of the image points must imply that this collection depicts a blood vessel having a geometric extent that is greater than a specified limit extent. Specifically, it is checked whether a diameter of this vessel, that represents a candidate for the arterial access position 17, is greater than a specified limit diameter. This limit diameter is selected in such a way that it corresponds to a conceivable minimum value for a diameter of an artery that is typically present and leads to the vascular structure 16.

The vein condition may only be met if a temporal condition is met, this being the case if at least one of the items of time information assigned to the respective image point specifies that the arrival time of the contrast agent at the corresponding position is greater than a venous time limit value. The venous time limit value, that may be permanently specified in principle, is imposed individually in the present case. With reference to the image data 12, the time point is therefore determined at which the contrast agent in the vascular structure 16 last arrived at a location at which a plurality of vessels come together, this time point representing the venous time limit value. With reference to FIG. 5, this check depicts that the venous condition is met for all image points that are present in the region of the venous access position 18.

Furthermore, in relation to the venous time limit value, the fact should also be considered that the time at which the contrast agent arrives at the venous access position may differ for various flow paths through the vascular structure 16. In order to counter difficulties that might arise from this in relation to checking the vein condition, it is possible to capture a plurality of items of time information for the image points, specifying the first arrival time of the contrast agent at the respective position specifically for different flow paths through the vascular structure 16. The evaluation of the temporal condition relating to the vein condition may take place separately for each of the flow paths. With regard to meeting the temporal condition of the vein condition, one important factor may be, for example, that item of time information, assigned to one of the flow paths, that specifies the greatest value for the arrival time of the contrast agent.

As for the checking of the artery condition, in relation to checking the vein condition of all image points for which the temporal condition is met as explained above, it is additionally checked whether a locality condition is met. All aspects explained in connection with the artery condition apply equally to the checking of the vein condition.

In the next step 25, with reference to the image data 12 or the graph 13, the processing device 6 establishes routes that lead from the access position 17, 18 to the peripheral position 15. For example, due to the existence of a plurality of access positions 17, 18 and a plurality of peripheral positions 15, the number of routes is typically correspondingly high. All of the routes represent potential access paths 19 for the catheter, in order to get from the respective access position 17, 18 to the respective peripheral position 15. An automated preselection relating to the routes that are established is carried out by the processing device 6 as explained below.

The automated preselection that is carried out by the processing device 6 in the respect of the established routes takes place in such a way that that those routes along whose course a diameter of the respective blood vessel is smaller than a specified minimum diameter and along whose course a curvature of the route is greater than a specified maximum curvature are eliminated. For this purpose, a corresponding suitability criterion is checked, that therefore relates to the question whether, on the basis of geometric conditions, the respective route is fundamentally suitable to be used as one of the access paths 19 for the catheter. The suitability criterion is therefore only met if the respective route is formed exclusively from blood vessels of the vascular structure whose diameters are greater than the specified minimum diameter. Furthermore, the suitability criterion is only met if the vessels forming the respective route do not at any location have a curvature that is greater than the specified maximum curvature.

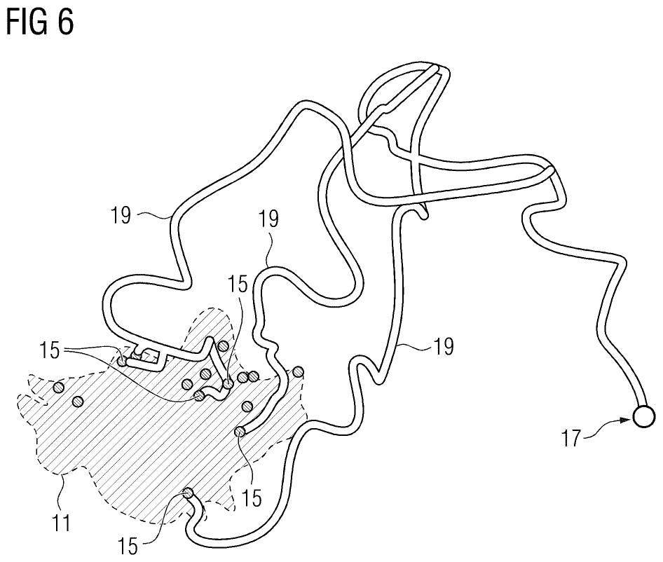

FIG. 6 depicts a perspective relating to the results that were established when executing the method explained above. On the right-hand side of this figure is indicated the arterial access position 17 that has been established. Additionally identifiable in the peripheral region of the pathological change 11 are the peripheral positions 15 that have been established, those routes that were not eliminated in the context of the preselection explained above being additionally depicted as access paths 19. The final decision as to which of the established routes are ultimately used when performing the embolization is made by the acting doctor.

A further aspect relating to the computer program 5 is explained in the following. In this case, the computer program 5 realizes an artificial intelligence in the form of a trained model that is used in the course of determining the access positions, the peripheral positions 15, the access paths and the segmentation. For the purpose of training the model, use is made of a plurality of sets including in each case training image data and respectively assigned training results.

It is to be understood that the elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present embodiments have been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for evaluating image data depicting a vascular structure for automatically determining at least one arterial and/or venous access position of the vascular structure, wherein an access path running from an access position and through the vascular structure leads to a respective peripheral position, the method comprising:
obtaining the image data comprising a plurality of image points, wherein the plurality of image points through whose respectively associated position in the vascular structure a contrast agent flowed during a recording of the image data, or of raw images from which the image data is derived, are each assigned at least one captured item of time information relating to a time point at which the contrast agent reached the respective position; and
determining the at least one arterial and/or venous access position based on the at least one captured item of time information.

2. The method of claim 1, wherein the image data or the raw images are captured using a medical imaging method using computed tomography and/or magnetic resonance tomography.

3. The method of claim 2, wherein an angular range of more than 180° is scanned by a C-arm for carrying out the computed tomography, wherein spatially three-dimensional image data or at least one spatially three-dimensional raw image is established from a set of two-dimensional raw images obtained from the medical imaging method.

4. The method of claim 1, wherein for generating the image data, the raw images are segmented and/or converted into at least one graph that models the vascular structure and comprises a collection of nodes and arcs.

5. The method of claim 4, wherein the raw images are segmented including where a region to be assigned to a pathological change is identified within the image data, wherein at least one node of the graph, the node being arranged in a peripheral region of the region, is identified as the respective peripheral position.

6. The method of claim 1, wherein for each of the image points whose corresponding position in the vascular structure is captured by the contrast agent, the method further comprises checking whether an artery condition is met, wherein the image points for which the artery condition is met are identified as an arterial access position or as belonging to an arterial access position, wherein the artery condition is met or may only be met if the item of time information or at least one of the items of time information implies that an arrival time of the contrast agent at the corresponding position is less than an arterial time limit value.

7. The method of claim 1, wherein for each of the image points whose corresponding position in the vascular structure is captured by the contrast agent, the method further comprises checking whether a venous condition is met, wherein the image points for which the venous condition is met are identified as a venous access position or as belonging to a venous access position, wherein the venous condition is met or may only be met if the item of time information or at least one of the items of time information implies that an arrival time of the contrast agent at the corresponding position is greater than a venous time limit value.

8. The method of claim 6, wherein the artery condition at an respective image point is only met or may only be met if, in addition to a temporal condition relating to the respective arrival time of the contrast agent, a locality condition is met which is only met or may only be met if the respective image point may be assigned to a collection of a plurality of image points, these being arranged directly adjacent to each other, for whom the temporal condition is likewise met, and if the collection of the image points implies that this represents a blood vessel having a geometric extent which is greater than a specified limit extent.

9. The method of claim 7, wherein the venous condition at an respective image point is only met or may only be met if, in addition to a temporal condition relating to the respective arrival time of the contrast agent, a locality condition is met which is only met or may only be met if the respective image point may be assigned to a collection of a plurality of image points, these being arranged directly adjacent to each other, for whom the temporal condition is likewise met, and if the collection of the image points implies that this represents a blood vessel having a geometric extent which is greater than a specified limit extent.

10. The method of claim 1, wherein a plurality of data sets comprising image data are obtained, wherein for obtaining the image data of one of the data sets, an injection of the contrast agent takes place via a blood vessel assigned to the respective data set, wherein the injection of the contrast agent takes place in different blood vessels for different data sets.

11. The method of claim 10, wherein for the image data of each of the data sets, the time point of the respective injection of the contrast agent is initially used as a reference time point for the at least one item of time information, wherein an image point which depicts a common location in the vascular structure is identified based on the image data of the data sets, wherein the item of time information assigned to the image point is used as a reference time point for the image data of the respective data set.

12. The method of claim 1, wherein at least one route that leads through the vascular structure and connects the respective access position to the respective peripheral position is established as the access path.

13. The method of claim 12, wherein a route that leads through the vascular structure and along whose course a diameter of a blood vessel concerned is greater than a specified minimum diameter, and/or a curvature of the route locally is smaller than a specified maximum curvature, is established as the access path.

14. A medical imaging device comprising:
at least one imaging unit that is configured to record and provide image data comprising a plurality of image points wherein the plurality of image points through whose respectively associated position in a vascular structure a contrast agent flowed during a recording of the image data, or of raw images from which the image data is derived, are each assigned at least one captured item of time information relating to a time point at which the contrast agent reached the respective position; and
at least one computing device that is configured to evaluate the image data depicting the vascular structure for automatically determining at least one arterial and/or venous access position of the vascular structure, wherein an access path running from an access position and through the vascular structure leads to a respective peripheral position, the at least one computing device configured to:
determine the at least one arterial and/or venous access position based on the at least one captured item of time information.

15. A non-transitory computer implemented storage medium, including machine-readable instructions stored therein for evaluating image data depicting a vascular structure for automatically determining at least one arterial and/or venous access position of the vascular structure, wherein an access path running from an access position and through the vascular structure leads to a respective peripheral position, the machine-readable instructions when executed by at least one processor, cause the processor to:
obtain the image data comprising a plurality of image points, wherein the plurality of image points through whose respectively associated position in the vascular structure a contrast agent flowed during a recording of the image data, or of raw images from which the image data is derived, are each assigned at least one captured item of time information relating to a time point at which the contrast agent reached the respective position; and
determine the at least one arterial and/or venous access position based on the at least one captured item of time information.
